(19) United States
(12) Patent Application Publication
(10) Pub. No.: US 2003/0155260 A1
(43) Pub. Date: Aug. 21, 2003

(54) Disposable footbath and handbath liner with or without herbal scents

(76) Inventors: Karin Maihart, Los Angeles, CA (US); Yvonne M. Spence, Santa Monica, CA (US)

Correspondence Address: KARIN MAIHART, 11931 MONTANA AVE., LOS ANGELES, LA, 90049, US

(21) Appl. No.: 10/080,068

(22) Filed: Feb. 20, 2002

(51) Int. Cl. A61L 15/00; A61B 19/02

(52) U.S. Cl. 206/438

(57) ABSTRACT

The invention provides a disposable, waterproof sheath that fits inside a footbath or hand/finger bowl to prevent germs from passing between uses and users of the bath or bowl.

### FIELD OF THE INVENTION

The invention is directed to disposable, waterproof foot and hand bath liners.

### SUMMARY OF THE INVENTION

This invention relates to new and useful improvements in sanitary inserts for the beauty industry but not limited to; beauty suppliers, cosmetology schools, hotels, spas, retreats, cruise ships, hospitals and everyday household use.

### BRIEF DESCRIPTION OF THE FIGURES

FIG. 1 is a photograph showing an embodiment of the sheath of the invention covering a hand bowl.

FIG. 2 is a photograph showing a sheath covering the handbowl without water and how a hand would fit the covered bowl.

FIG. 3 is a photograph showing a sheath covering a hand bowl with a hand immersed in fluid.

FIG. 4 is a photograph showing a footbath without a sheath

FIG. 5 is a photograph showing a footbath with its inner portion covered with a sheath of the invention.

FIG. 6 is a photograph showing feet immersed in fluid in the sheath-covered footbath.

### DETAILED DESCRIPTION OF THE INVENTION

The present invention generally relates to a footbath and hand/finger and nail bowl sheath/insert/liner for placement within footbaths and/or nail and finger bowls especially footbaths with a motors which carry germs. The insert which is disposable and can be replaced after a single use provides a sanitary condition and will eliminate infection, disease, germs and fungus that typically spreads as a result of multiple use by multiple users of a foot bath or hand bowl.

The disposable sheath can be scented or unscented. Further, it allows clean water to fill the container without exposing the water or a person's nails or feet to an unsanitary condition.

The sheath/insert/liner is a single sheath/sheet of thin yet tough, flexible, waterproof, material (typically plastic) that may be of any suitable or desirable shape and size and specifically fits the general contour as the inner contour of the footbath and/or a nail and finger bowl.

The depth of the insert will depend on the area and width—greater or lessor for expansion of the sheath/insert/liner.

As an example the depth of the footbath is deeper that that of

The finger/nail bowl. For example, a sheath for a foot bath may measure 14″wide×16″deep×8″high, or 13″wide×15″deep×6″ high Additionally, m accordance with the invention a sheath for a hand bowl may measure 4½″wide×6″long×2″ high. However, since bowls and baths may differ in size a sheath of any size can be constructed and will be encompassed by the invention.

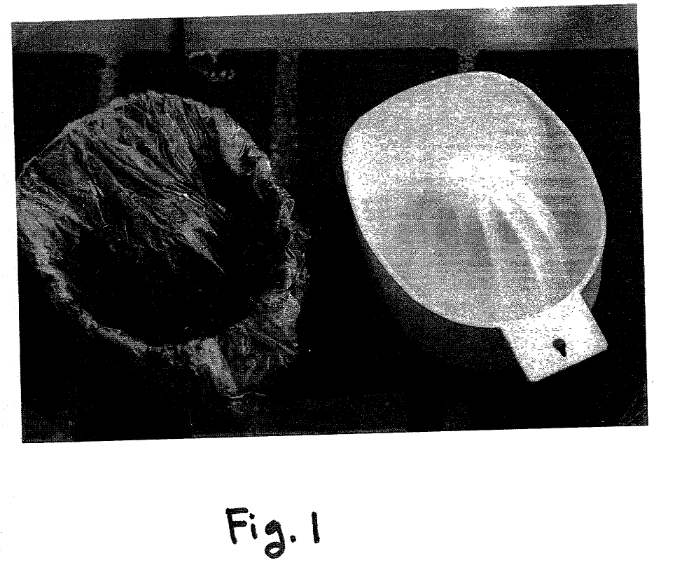

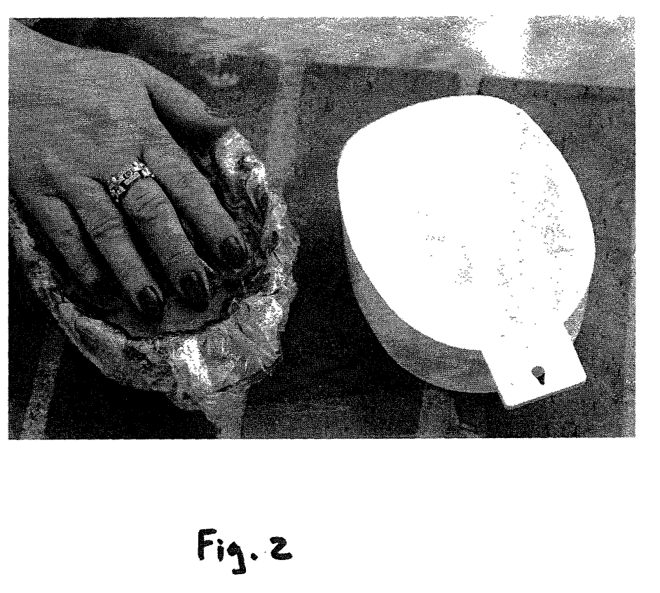

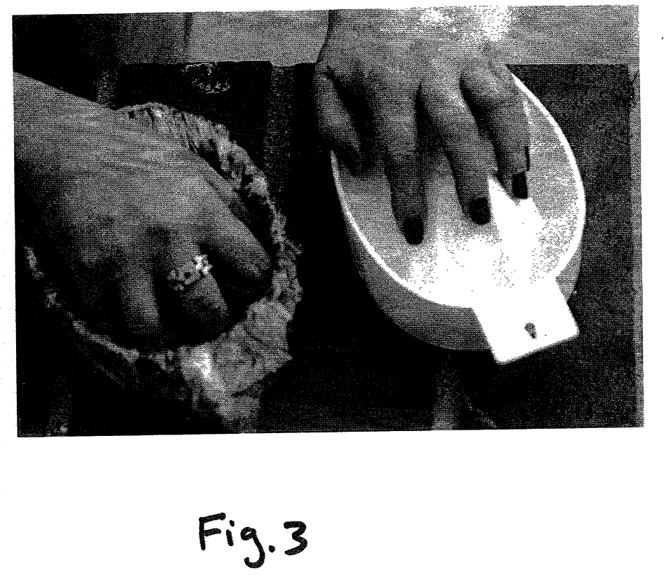

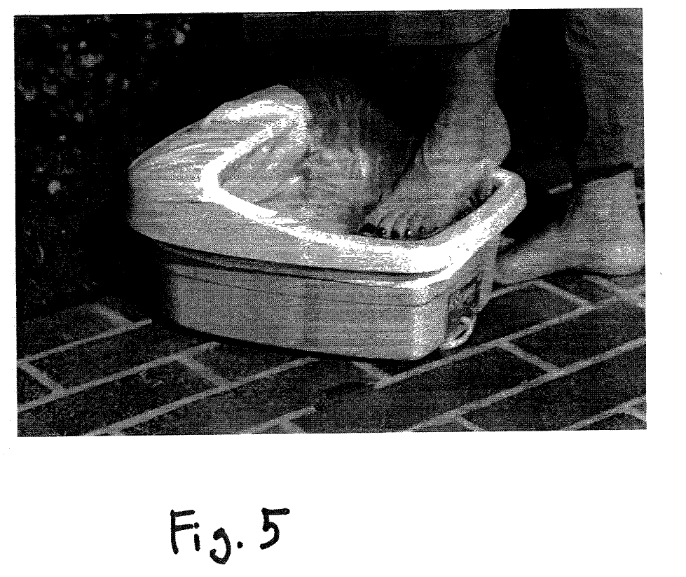

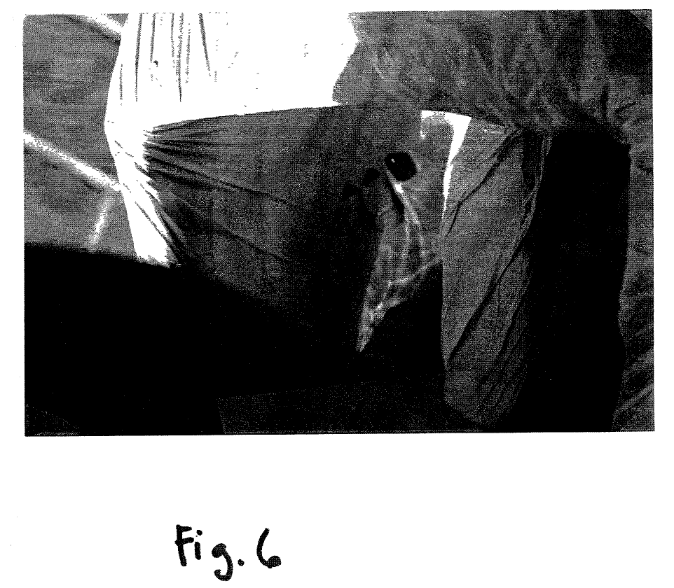

While the securing of the edges of this sheath/insert/liner may be accomplished in various ways. We have here by shown one particular way of doing it and that is by using elastic as shown in FIGS. 1-6.

The walls of the sheath/insert/liner preferably should snuggly conform or adhere to the sides of the footbath or finger and nail bowl

The inside of the sheath/insert/liner can be part of kit that includes a pouch having herbs, salts, or oils to be used during the foot/hand treatment. The pouch can include tear off tabs allowing the herbal scents/salts/oils to dissipate in the water.

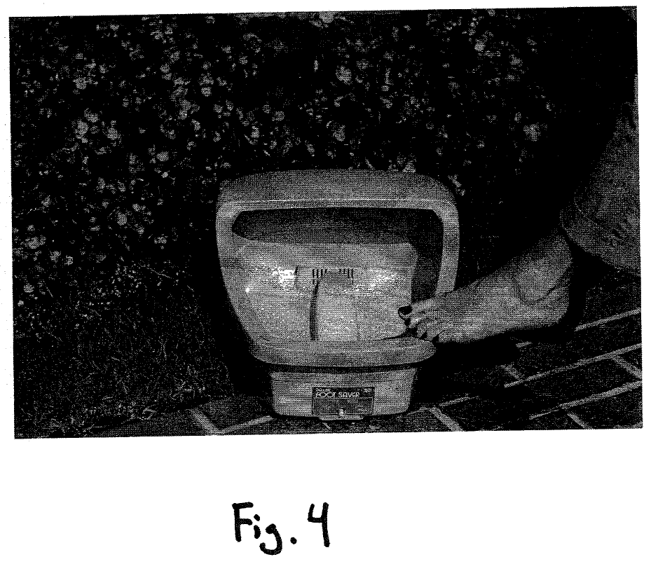

The edges of the footbath are ready to receive water as shown in FIGS. 4-6.

In addition, the sheath/insert/liner may have not restricted to a saying or a hotel name written on them.

After all, we only have 10 fingers and 10 toes . . . and we need those!

The previous description is provided to assist one of ordinary skill in making and using the invention. The descriptions above are not intended in any way to otherwise limit the scope of the disclosure of the protection granted by Letters Patent granted hereon.

What is claimed is:

1. A disposable waterproof sheath to cover an inner portion of a container in order to protect an appendage from microbial matter in the inner container, the sheath being a) deep enough so that the appendage can enter the container and b) conforms to the edge of the container so that the sheath adheres to the container edge or grips the container edge and prevents the appendage from contacting the inner portion of the container.

2. The disposable, waterproof and sheath of claim 1, wherein the appendage is a portion of an arm, a hand, a finger, a portion of a leg, a foot, or a toe.

3. A kit comprising the disposable waterproof sheath of claim 1 and scented herbs.

4. A kit comprising the disposable waterproof sheath of claim 1 and bath salts.

5. A kit comprising the disposable waterproof sheath of claim 1 and bath oils.

6. The kit of claim 4, wherein the bath salts are Epson salts.
